(19) United States
(12) Patent Application Publication
(10) Pub. No.: US 2019/0078099 A1
(43) Pub. Date: Mar. 14, 2019

(54) CELL LINE FOR RECOMBINANT PROTEIN AND/OR VIRAL VECTOR PRODUCTION

(71) Applicant: SPARK THERAPEUTICS, INC., Philadelphia, PA (US)

(72) Inventors: Jingmin ZHOU, Wallingford, PA (US); Guang QU, Sicklerville, NJ (US); John Fraser WRIGHT, Princeton, NJ (US)

(73) Assignee: SPARK THERAPEUTICS, INC., Philadelphia, PA (US)

(21) Appl. No.: 16/088,693

(22) PCT Filed: Mar. 30, 2017
(86) PCT No.: PCT/US17/24951

§ 371 Date: Sep. 26, 2018

Related U.S. Application Data
(60) Provisional application No. 62/315,480, filed on Mar. 30, 2016.

(51) Int. Cl. C12N 15/52; C07K 14/705; C12N 15/85; C12N 9/06; C12N 9/22; C12N 9/00; C12N 9/12; C12N 15/62

(52) U.S. Cl. CPC C12N 15/52; C07K 14/705; C12N 15/85; C12N 9/003; C12N 9/22; C12N 9/93; C12Y 207/07042; C12N 15/62; C12Y 105/01003; C12N 2750/14143; C12N 2750/14152; C12N 2015/8518; C12N 9/1241

(57) ABSTRACT

Cells and cell lines are disclosed that are able to produce therapeutic proteins, antibodies, vectors, and viral vectors such as lentiviral vectors and adeno-associated viral (AAV) vectors. The cells and/or cell lines can have mutations or deletions in either one or both of the endogenous di-hydrofolate reductase (DHFR−/−) or glutamine synthetase (GS−/−) genes such that DHFR and/or GS expression or function is substantially reduced or eliminated.

### RELATED APPLICATIONS

This application claims priority to U.S. Provisional Patent Application No. 62/315,480, filed Mar. 30, 2016. The entire contents of the foregoing application is incorporated herein by reference, including all text, tables, sequence listing and drawings.

### INTRODUCTION

Glutamine synthetase (GS) is an enzyme in the synthesis of the amino acid L-glutamine A GS-negative cell line is therefore auxotrophic for L-glutamine. GS has been reported as a selection marker gene in CHO cell based recombinant protein expression systems (Wurm et al. (2004) Nature Biotechnology 22: 1393-1398). An expression cassette containing GS gene can be selected using GS inhibitor methionine sulfoximine when the cassette is introduced into a GS-negative CHO line.

Dihydrofolate reductase (DHFR, 5,6,7,8-tetrahydrofolate:NADP+oxidoreductase) is an enzyme in both eukaryotes and prokaryotes and catalyzes the NADPH-dependent reduction of dihydrofolate to tetrahydrofolate, an essential carrier of one-carbon units in the biosynthesis of thymidylate, purine nucleotides, glycine and methyl compounds. DHFR-deficient cells will only grow in medium supplemented by certain factors involved in folate metabolism or if DHFR is provided to the cell, for example as a transgene.

### SUMMARY

Cells and cell lines are disclosed herein that are able to produce therapeutic proteins, antibodies, vectors, and viral vectors such as lentiviral vectors and adeno-associated viral (AAV) vectors. The cells and/or cell lines can have mutations or deletions in either one or both of the endogenous dihydrofolate reductase (DHFR−/−) or glutamine synthetase (GS−/−) genes such that DHFR and/or GS expression or function is substantially reduced or eliminated.

Reduction can be achieved, for example, by a single allele knockout of DHFR and/or GS gene(s). Reduction can be achieved by a mutation (e.g., substitution or deletion) in a DHFR and/or GS gene(s) that reduces function or activity of the corresponding protein. Elimination can be achieved by a bi-allele knock-out of DHFR and/or GS gene(s).

In particular embodiments, invention cells and/or cell lines are based upon or derived from human embryonic kidney (HEK) cells or cell lines, such as HEK293. Human embryonic kidney (HEK) cells or cell lines, such as HEK293, as disclosed herein have mutations or deletions in either one or both of the endogenous dihydrofolate reductase (DHFR−/−) or glutamine synthetase (GS−/−) genes such that DHFR and/or GS protein expression and/or function is substantially reduced or eliminated.

In particular embodiments, invention cells and/or cell lines are based upon or derived from human adenocarcinoma alveolar basal epithelial cells or cell lines. Human A459 cells or cell lines, as disclosed herein have mutations or deletions in either one or both of the endogenous dihydrofolate reductase (DHFR−/−) or glutamine synthetase (GS−/−) genes such that DHFR and/or GS protein expression and/or function is substantially reduced or eliminated.

In particular embodiments, invention cells and/or cell lines are based upon or derived from kidney of an African green monkey. Vero cells or cell lines, as disclosed herein have mutations or deletions in either one or both of the endogenous dihydrofolate reductase (DHFR−/−) or glutamine synthetase (GS−/−) genes such that DHFR and/or GS protein expression and/or function is substantially reduced or eliminated.

Cell lines can be selected from individual cells (clones). The clones can be expanded and in turn can provide a stable cell line of HEK cells, such as HEK293, human A459 cells and/or Vero cells, with the mutations or deletions in either one or both of the endogenous dihydrofolate reductase (DHFR−/−) or glutamine synthetase (GS−/−) genes such that DHFR and/or GS expression and/or function is substantially reduced or eliminated.

Disclosed herein, in some aspects, is a human embryonic kidney (HEK) cell, a human A459 cell and a Vero cell which does not express a functional endogenous di-hydrofolate reductase (DHFR) and/or glutamine synthetase (GS). In certain aspects, presented herein is a human embryonic kidney (HEK) cell line, human A459 cell line and a Vero cell line that does not express a functional endogenous di-hydrofolate reductase (DHFR) and/or glutamine synthetase (GS).

In some embodiments, the HEK cell or cell line, human A459 cell or cell line and/or Vero cell or cell line is stably or transiently transfected with a first heterologous nucleic acid sequence, and optionally stably or transiently transfected with a second heterologous nucleic acid sequence. In some embodiments, the HEK cell or cell line, human A459 cell or cell line and/or Vero cell or cell line is stably or transiently transfected with the first heterologous nucleic acid sequence and a first selectable marker, and optionally stably or transiently transfected with the second heterologous nucleic acid sequence and a second selectable marker. In certain embodiments, the first heterologous nucleic acid sequence encodes a therapeutic protein or polynucleotide sequence and, in certain embodiments, the second heterologous nucleic acid sequence encodes a therapeutic protein or polynucleotide sequence. The therapeutic protein or polynucleotide sequence encoded by the first heterologous nucleic acid sequence and the therapeutic protein or polynucleotide sequence encoded by the optional second heterologous nucleic acid sequence can be the same or different. In some embodiments, the first and/or second selectable marker does not provide resistance to an antibiotic. In certain embodiments, the first and/or second selectable marker provides a means to amplify the first and/or second heterologous nucleic acid sequence(s). In some aspects, the first and/or second selectable marker comprises a nucleic acid encoding a protein having DHFR function. In some aspects, the first and/or second selectable marker comprises a nucleic acid encoding a protein having GS function. In some embodiments, the first selectable marker comprises a nucleic acid encoding a protein having DHFR function and the second selectable marker comprises a nucleic acid encoding a protein having GS function.

In some aspects of the HEK cell or cell line, human A459 cell or cell line and/or Vero cell or cell line described herein, the first heterologous nucleic acid sequence comprises a first vector, and, in some embodiments, the optional second heterologous nucleic acid sequence comprises a second vector. The first vector and optional second vector can be the same or different. In some embodiments, the first vector and optional second vector each comprises a selectable marker comprising a nucleic acid encoding a protein having DHFR function or a nucleic acid encoding a protein having GS function. In certain embodiments, the first vector comprises a first viral vector and optional second vector comprises a second viral vector. In some embodiments, the first and/or second viral vector comprises an AAV vector genome. In some embodiments, where the HEK cell or cell line, human A459 cell or cell line and/or Vero cell or cell line comprises a first and second viral vector, each of the viral vectors comprise an AAV vector genome, or portion thereof. In certain embodiments, the AAV vector genome(s) comprises one or two AAV ITRs that flank the 5′ and/or 3′ ends of the heterologous nucleic acid sequence.

In certain aspects, a copy number of the heterologous nucleic acid sequence(s) and/or vector(s) and/or viral vector(s) and/or AAV vector genome(s) in the HEK cell or cell line, human A459 cell or cell line and/or Vero cell or cell line is between 10 and 5000 copies per cell. In some embodiments, a copy number of the heterologous nucleic acid sequence(s) and/or vector(s) and/or viral vector(s) and/or AAV vector genome(s) in the HEK cell or cell line, human A459 cell or cell line and/or Vero cell or cell line is between 1-5 copies per cell, 5-10 copies per cell, 10-50 copies/cell, 50-100 copies per cell, 100-250 copies per cell, 250-500 copies per cell, 500-1,000 copies per cell, 1,000-2,000 copies per cell, or about or greater than 2,000, 3,000, 4,000 or 5,000 copies per cell. In certain embodiments, the copy number of the AAV vector genome(s) in the HEK cell or cell line, human A459 cell or cell line and/or Vero cell or cell line is at least 1,000 copies per cell, and the rAAV vector particle yield is at least 1×10⁸ vg/ml, at least 1×10⁹ vg/ml, at least 1×10¹⁰ vg/ml, at least 1×10¹¹ vg/ml or at least 2×10¹¹ vg/ml from Roller Bottle of HEK cells or of the HEK cell line, human A459 cells or cell line and/or Vero cells or cell line. In some embodiments, copy number appears stable over many passages, e.g., at least or greater than 5, 10, 15, 20, 30, 40, 50, or more passages and AAV vector production is stable and consistent, for example, within about 10-30% of the amount produced from any fewer cell passages.

In some aspects, the HEK cell or cell line, human A459 cell or cell line and/or Vero cell or cell line presented herein further comprises AAV rep and/or cap sequences. In some embodiments, the AAV rep and/or cap sequences are provided by a plasmid that is either transiently or stably transfected into the HEK cell or cell line, human A459 cell or cell line and/or Vero cell or cell line. In some embodiments, the HEK cell or cell line, human A459 cell or cell line and/or Vero cell or cell line presented herein further comprises AAV helper function sequences.

In certain aspects, the HEK cell or HEK cell line presented herein is HEK 293.

In some embodiments, the HEK cell or cell line, human A459 cell or cell line and/or Vero cell or cell line presented herein is in a culture or growth medium or in a medium suitable for long-term storage. In some embodiments, the culture medium or growth comprises methotrexate (MTX) and/or methionine sulphoxamine (MSX).

In certain aspects, the HEK cell or cell line, human A459 cell or cell line and/or Vero cell or cell line presented herein produces rAAV vector particles having packaged therein one or more heterologous nucleic acid sequence(s) (e.g., a first and/or a second heterologous nucleic acid as described herein). In some embodiments, the rAAV vector particles are produced in greater amounts than amounts produced by HEK293 cells that express functional endogenous DHFR and/or GS and transiently transfected with AAV vector genomes having the heterologous nucleic acid sequence. In certain embodiments, the AAV vector particles produced contain less quantities (e.g., at least 1%, at least 10% less or at least 2-fold less) of rAAV empty capsids and/or less quantities (e.g., at least 1%, at least 10% less or at least 2-fold less) of rAAV particles that have packaged contaminating DNA than amounts of AAV empty capsid and/or rAAV particles that have packaged contaminating DNA produced by HEK293 cells that express functional endogenous DHFR and/or GS and transiently transfected with rAAV vector genomes having the heterologous nucleic acid sequence.

In certain aspects, heterologous nucleic acid sequence(s) encodes one or more therapeutic protein(s). In certain aspects, heterologous nucleic acid sequence(s) encodes one or more inhibitory factors. In some embodiments, a heterologous nucleic acid sequence(s) comprises one or more inhibitory nucleic acid sequence(s). In some embodiments, a heterologous nucleic acid sequence(s) encodes a therapeutic protein(s) and/or comprises an inhibitory nucleic acid sequence(s). In certain embodiments, the therapeutic protein(s) comprises a blood clotting factor. In certain embodiments, the therapeutic protein(s) comprises a immunoglobulin sequence (e.g., an amino acid sequence of an immunoglobulin). In some embodiments, an inhibitory nucleic acid sequence comprises a small or short hairpin (sh)RNA, microRNA (miRNA), small or short interfering (si)RNA, trans-splicing RNA, or antisense RNA.

In certain aspects, the HEK cell or cell line, human A459 cell or cell line and/or Vero cell or cell line described herein is stably transfected with the first heterologous nucleic acid sequence. In some embodiments, the HEK cell or cell line, human A459 cell or cell line and/or Vero cell or cell line described herein is stably transfected with the first and/or the second heterologous nucleic acid sequence.

In some aspects, presented herein are viral or rAAV vector particles isolated and/or purified from an HEK cell or cell line, human A459 cell or cell line and/or Vero cell or cell line described herein.

In some aspects, presented herein are therapeutic protein(s) isolated and/or purified from a HEK cell or cell line, human A459 cell or cell line and/or Vero cell or cell line described herein.

In some aspects, presented herein is a method of producing a therapeutic protein(s), viral vector(s) and/or rAAV vector particles, the method comprising culturing an HEK cell or cell line, human A459 cell or cell line and/or Vero cell or cell line described herein, under conditions allowing production and/or secretion of the therapeutic protein(s), viral vector(s) or rAAV vector particles described herein, and isolating or purifying the therapeutic protein(s), viral vector(s) or rAAV vector particles from a cell culture, culture medium, or cell culture and culture medium (e.g., a cell culture, culture medium, or cell culture and culture medium comprising the HEK cell or cell line, human A459 cell or cell line and/or Vero cell or cell line).

In some aspects, presented herein are methods of producing rAAV vector particles comprising culturing an HEK cell or cell line, human A459 cell or cell line and/or Vero cell or cell line described herein, under conditions allowing production and/or secretion of the rAAV vector particles and isolating or purifying the rAAV vector particles from the cell culture, culture medium, or cell culture and culture medium, where the HEK cell or cell line, human A459 cell or cell line and/or Vero cell or cell line has at least 1,000 copies per cell of AAV vector genome, the rAAV vector particle yield is at least 1×10⁸ vg/ml, or at least 1×10⁹ vg/ml, or at least 1×10¹⁰ vg/ml, or at least 1×10¹¹ vg/ml or at least 2×10¹¹ vg/ml from Roller Bottle of the HEK cell or the HEK cell line, human A459 cell or cell line and/or Vero cell or cell line.

In some aspects, a first and/or second heterologous nucleic acid sequence described herein encodes a gene product selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), TGFβ, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase.

In certain aspects, presented herein are a HEK cell or cell line, human A459 cell or cell line and/or Vero cell or cell line a described herein, or a method as described herein, where the rAAV vector particles comprise the first and/or the second heterologous nucleic acid sequence encoding a gene product selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), TGFβ, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase.

In certain aspects, presented herein are a HEK cell or cell line, human A459 cell or cell line and/or Vero cell or cell line a described herein, or a method as described herein, where the first and/or second heterologous nucleic acid sequence encodes a gene product selected from the group consisting of thrombopoietin (TPO), interleukins (IL1 through IL-17), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

In certain aspects, presented herein are a HEK cell or cell line, human A459 cell or cell line and/or Vero cell or cell line a described herein, or a method as described herein, where the rAAV vector particles comprise the first and/or the second heterologous nucleic acid sequence encoding a gene product selected from the group consisting of thrombopoietin (TPO), interleukins (IL1 through IL-17), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

In certain aspects, presented herein are a HEK cell or cell line, human A459 cell or cell line and/or Vero cell or cell line a described herein, or a method as described herein, where the first and/or second heterologous nucleic acid sequence encodes a protein useful for correction of in born errors selected from the group consisting of carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor V, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, RPE65, H-protein, T-protein, a cystic fibrosis transmembrane regulator (CFTR) sequence, and a dystrophin cDNA sequence.

In certain aspects, presented herein are a HEK cell or cell line, human A459 cell or cell line and/or Vero cell or cell line a described herein, or a method as described herein, where the rAAV vector particles comprise the first and/or the second heterologous nucleic acid sequence encoding encodes a protein useful for correction of in born errors selected from the group consisting of carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor V, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, RPE65, H-protein, T-protein, a cystic fibrosis transmembrane regulator (CFTR) sequence, and a dystrophin cDNA sequence.

In some embodiments, presented herein are methods of producing a human embryonic kidney (HEK) cell line, human A459 cell line and Vero cell line which does not express functional endogenous di-hydrofolate reductase (DHFR), the method comprising mutating or knocking out an endogenous DHFR gene.

In some embodiments, presented herein are methods of producing a human embryonic kidney (HEK) cell line, human A459 cell line and Vero cell line which does not express functional endogenous glutamine synthetase (GS), comprising mutating or knocking out the endogenous GS gene.

In some embodiments, presented herein are methods of producing a human embryonic kidney (HEK) cell line, human A459 cell line and Vero cell line which does not express functional endogenous di-hydrofolate reductase (DHFR) and glutamine synthetase (GS), comprising mutating or knocking out the endogenous DHFR gene and GS gene.

### BRIEF DESCRIPTION OF DRAWINGS

FIG. 1 shows a brief overview of the creation of the human embryonic kidney (HEK) cell or cell line, such as HEK293, and use for producing recombinant proteins and viral vectors such as AAV.

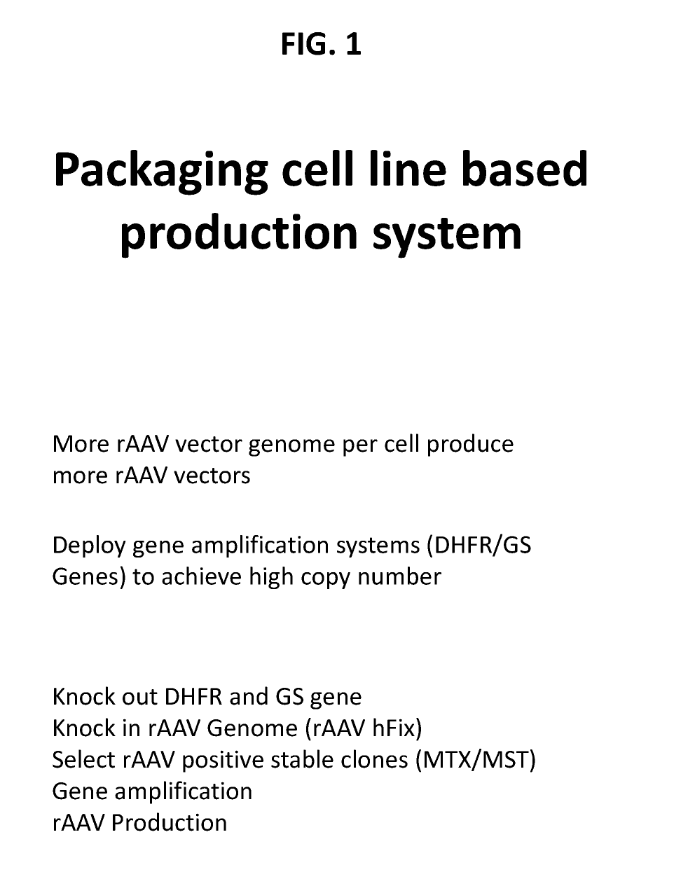

FIG. 2 shows amounts of rAAV vector produced by exemplary HEK293 clones (stable cell lines) of the invention. Y-axis shows AAV vector (vector genomes, vg) produced by each HEK293 clone in a roller bottle.

### DETAILED DESCRIPTION

Clones of cells with such gene(s) modified or gene(s) knocked out of HEK cells, such as HEK293 cells, are the first clones of human cells with DHFR−/— and/or GS−/− genomic background. These cells and cell lines can be used to produce many different recombinant biomaterials, such as recombinant proteins (e.g., antibodies such as monoclonal antibodies) and viral vectors. The recombinant proteins and viral vectors produced can be used for treatment of diseases. In particular applications, viral vectors (e.g., lenti- or AAV) produced can be used for a knock-in (e.g., introduce a functional protein which is aberrant or missing) gene therapy application. In particular applications, viral vectors (e.g., lenti- or AAV) produced can be used for a knock out (e.g., introduce an inhibitory sequence such as an antisense to target an endogenous protein whose expression or function is aberrant or undesired, such as a mutant protein that causes or is associated with a pathologies or diseases) gene therapy application.

This invention will provide benefit to production of biologicals, such as proteins, and other bio-materials, including recombinant proteins, antibodies, viral vectors including AAV, lenti- and other viruses, by way of a gene amplification system in a well characterized human cell line. Additional benefit is for the production of proteins that require a human intracellular environment for folding, modification (post-translational) and function.

This invention creates a new production system using well characterized human cells or cell lines. The parental clones selected to establish rAAV producing cell lines are engineered from HEK (e.g., HEK293) cells, human A459 cells or Vero cells with substantially reduced or eliminated DHFR and/or GS genes, such as a single- or double-knock out of DHFR and/or GS genes. The human HEK (e.g., HEK293), human A459 and/or Vero cells and cell lines with substantially reduced or eliminated DHFR and/or GS genes, e.g., single- or double-knock out of DHFR and/or GS genes of this invention will enable post-translational modification of the bio-products more closely to its natural modification in human, and therefore improve the safety and bioactivity of the bio-products.

Eliminating (e.g., knocking out) one or both DHFR and GS genes creates one or two selection markers for the HEK293 cells, human A459 cells and/or Vero cells. Invention HEK cells and cell lines, human A459 cells and cell lines and/or Vero cells and cell lines into which a DHFR selectable marker has been stably integrated can be selected for by culturing the cells in a culture medium. Moreover, a heterologous nucleic acid sequence separate from a DHFR selectable marker (e.g., two separate plasmids) or as a single polynucleotide sequence (e.g., on the same plasmid, such as an AAV vector plasmid) can both be integrated when introduced into a cell. Accordingly, when the DHFR transgene has added or includes a heterologous nucleic acid sequence (e.g., encoding a protein or nucleic acid of interest), cells can be selected that express both DHFR and the protein or nucleic acid of interest.

Invention HEK cells and cell lines, human A459 cells and cell lines and/or Vero cells and cell lines into which a GS selectable marker has been stably integrated can be selected for by culturing the cells in a culture medium. A heterologous nucleic acid sequence separate from a GS selectable marker (e.g., two separate plasmids) or as a single polynucleotide sequence (e.g., on the same plasmid, such as an AAV vector plasmid) can both be integrated when introduced into a cell. Thus, when the GS transgene has added or includes a heterologous nucleic acid sequence (e.g., encoding a protein or nucleic acid of interest), cells can be selected that express both GS and the protein or nucleic acid of interest.

In response to inhibitors such as methotrexate (MTX), the DHFR gene copy number, and therefore the heterologous nucleic acid sequence integrated proximally to the DHFR gene can be amplified in invention HEK cells and cell lines, human A459 cells and cell lines and/or Vero cells and cell lines. In response to inhibitors such as methionine sulfoximine (MS) the GS gene copy number, and therefore the heterologous nucleic acid sequence integrated proximally to the GS gene can be amplified in invention HEK cells and cell lines, human A459 cells and cell lines and/or Vero cells and cell lines.

Accordingly, sequences encoding a protein or nucleic acid sequences of interest of interest that are integrated proximally to or co-integrated with exogenous DHFR and/or GS can be amplified by gradually exposing the cells to increasing concentrations of MTX and/or MS, resulting in increased expression of the encoded protein or nucleic acid of interest.

Studies disclosed herein show that HEK293 clones were obtained that have high copy numbers of rAAV genome. In a particular example, the rAAV genome encodes therapeutic human FIX, the copies of the rAAV genomes reached to the level of more than thousand copies which is significantly higher than in most stable cell lines reported. The high copy number of rAAV genome resulted in high rAAV-hFix production. The high copy number appears stable over many passages, e.g., at least or greater than 5, 10, 15, 20 30, 40, 50, or more passages.

In addition to high rAAV production, the quality of the rAAV preps produced from the HEK293 rAAV vector producing cell lines were also evaluated. It appears that high rAAV genome copies in HEK293 stable clones may further reduce DNA impurities packaged in the rAAV particles and reduce the ratio of empty particles relative to genome packed vectors.

In addition to the high copies of rAAV genome in the stable producing HEK293 clones, the invention HEK (e.g., HEK293) cells and cell lines, human A459 cells and cell lines and/or Vero cells and cell lines also enable gene amplification through induction of MTX and/or MSX, which will amplify rAAV genome further in the HEK (e.g., HEK293) cells and cell lines, human A459 cells and cell lines and/or Vero cells and cell lines. Amplification will in turn increase number of rAAV genome thereby increasing rAAV production by the engineered HEK (e.g., HEK293) cells and cell lines, human A459 cells and cell lines and/or Vero cells and cell lines.

Invention HEK cells or cell lines, human A459 cells and cell lines and/or Vero cells and cell lines, such as knock in clones of HEK cells (HEK293 single DFHR−/− or GS−/− or, double DFHR−/−/GS−/−) can be used to produce rAAV vectors of any AAV serotype. For example, invention HEK cells or cell lines, human A459 cells or cell lines and/or Vero cells or cell lines, such as viral (e.g., AAV) vector knock in clones of HEK293 rAAV-hFix DFHR−/−/GS−/− can be used to produce rAAV-hFix vectors of any AAV serotype, and the vector produced can be used for treatment of Hemophilia B by way of gene therapy.

Particular non-limiting methods in which the invention HEK cells or cell lines can be used to manufacture viral (e.g., AAV) vectors are described in US 2013/0072548 (U.S. Ser. No. 13/561,753); US 2014/0349403 (U.S. Ser. No. 14/364,623); and US 2014/0323556 (U.S. Ser. No. 14/216,778).

A “selectable marker” refers to a polynucleotide or gene which when introduced and when expressed by cells, under appropriate selective culture conditions, allows for the selection of cells expressing said selectable marker. A selectable marker can be DHFR and/or GS, in particular a polynucleotide or gene that encodes a protein having DHFR and/or GS function or activity, such as a DHFR and/or GS protein expressed by or in invention HEK (e.g., HEK293) cells and cell lines, human A459 cells and cell lines and/or Vero cells and cell lines.

All mammalian and non-mammalian forms of DHFR proteins and GS proteins and encoding nucleic acids are expressly included. Suitable DHFR proteins and GS proteins and accordingly genes known to one skilled in the art can be used. DHFR and GS proteins can be derived from any species as long as it retains at least partial function or activity in invention HEK (e.g., HEK293) cells and cell lines, human A459 cells and cell lines and/or Vero cells and cell lines. DHFR and/or GS proteins include naturally occurring polymorphic forms.

The DHFR and/or GS may be a wildtype DHFR and/or GS or a functional variant or derivative thereof. The term “variant” or “derivative” includes DHFR and/or GS proteins (the term can also refer to the nucleic acid sequence encoding such proteins) having one or more amino acid sequence exchanges (e.g. deletions, substitutions or additions) with respect to the amino acid sequence of the respective DHFR and/or GS protein, fusion protein comprising a DHFR and/or GS protein or functional fragment thereof. Variants include DHFR and/or GS protein(s) that retain at least partial function or activity of DHFR and/or GS protein(s).

Variants and derivatives of DHFR and/or GS proteins which have been modified to provide an additional structure and/or function, as well as functional fragments of the foregoing, which still have at least one function of a DHFR and/or GS protein. For example, a DHFR and/or GS protein may be used as selectable maker that is e.g. more or less sensitive to antifolates such as MTX or more or less sensitive to MS than a wildtype DHFR or GS protein, respectively, and/or the DHFR or GS protein endogenously expressed by the HEK (e.g., HEK293) cells, human A459 cells and/or Vero cells.

For example, a DHFR protein used as selectable marker which is more susceptible to a DHFR inhibitor such as MTX than endogenous DHFR enzyme expressed in invention HEK (e.g., HEK293) cells and cell lines, human A459 cells and cell lines and/or Vero cells and cell lines. Such a DHFR in turn provides a means for robust amplification of DHFR selectable marker and in turn the heterologous nucleic acid/vector sequences.

The term “vector” refers to small carrier nucleic acid molecule, a plasmid, virus (e.g., AAV vector), or other vehicle that can be manipulated by insertion or incorporation of a nucleic acid. Vectors can be used for genetic manipulation (i.e., “cloning vectors”), to introduce/transfer polynucleotides into cells, and to transcribe or translate the inserted polynucleotide in cells. An “expression vector” is a vector that contains a gene or nucleic acid sequence with the necessary regulatory regions needed for expression in a host cell. A vector nucleic acid sequence generally contains at least an origin of replication for propagation in a cell and optionally additional elements, such as a heterologous nucleic acid sequence, expression control element (e.g., a promoter, enhancer), intron, inverted terminal repeats (ITRs), optional selectable marker (e.g., DHFR, GS, etc.), polyadenylation signal.

A viral vector is derived from or based upon one or more nucleic acid elements that comprise a viral genome. Particular viral vectors include lentivirus, pseudo-typed lentivirus and parvo-virus vectors, such as adeno-associated virus (AAV) vectors. Parvoviruses including AAV are useful as gene therapy vectors as they can penetrate cells and introduce nucleic acid/genetic material so that the nucleic acid/genetic material may be stably maintained in cells. In addition, these viruses can introduce nucleic acid/genetic material into specific sites, for example, such as a specific site on chromosome 19. Because AAV are not associated with pathogenic disease in humans, AAV vectors are able to deliver heterologous nucleic acid sequences (e.g., therapeutic proteins and agents) to human patients without causing substantial AAV pathogenesis or disease.

The term “recombinant,” as a modifier of vector, such as recombinant viral, e.g., lenti- or parvo-virus (e.g., rAAV) vectors, as well as a modifier of sequences such as recombinant polynucleotides and polypeptides, means that the compositions have been manipulated (i.e., engineered) in a fashion that generally does not occur in nature. A particular example of a recombinant vector, such as an AAV vector would be where a polynucleotide that is not normally present in the wild-type viral (e.g., AAV) genome is inserted within the viral genome. An example of a recombinant vector would be where a nucleic acid (e.g., gene) encoding a therapeutic protein or polynucleotide sequence is cloned into a vector, with or without 5′, 3′ and/or intron regions that the gene is normally associated within the viral (e.g., AAV) genome. Although the term “recombinant” is not always used herein in reference to vectors, such as viral and AAV vectors, as well as sequences such as polynucleotides, recombinant forms including polynucleotides, are expressly included in spite of any such omission.

A recombinant viral “vector” or “rAAV vector” is derived from the wild type genome of a virus, such as AAV by using molecular methods to remove the wild type genome from the virus (e.g., AAV), and replacing with a non-native (heterologous) nucleic acid, such as a nucleic acid encoding a therapeutic protein or polynucleotide sequence. Typically, for AAV, inverted terminal repeat (ITR) sequences of AAV genome are retained in the rAAV vector. A “recombinant” viral vector (e.g., rAAV) is distinguished from a viral (e.g., AAV) genome, since all or a part of the viral genome has been replaced with a non-native sequence with respect to the viral (e.g., AAV) genomic nucleic acid such as a heterologous nucleic acid encoding a therapeutic protein or polynucleotide sequence. Incorporation of a non-native sequence therefore defines the viral vector (e.g., AAV) as a “recombinant” vector, which in the case of AAV can be referred to as a “rAAV vector.”

A recombinant vector (e.g., lenti-, parvo-, AAV) sequence can be packaged-referred to herein as a “particle” for subsequent infection (transduction) of a cell, ex vivo, in vitro or in vivo. Where a recombinant vector sequence is encapsidated or packaged into an AAV particle, the particle can also be referred to as a “rAAV.” Such r AAV particles include proteins that encapsidate or package the vector genome. Particular examples include viral envelope proteins, and in the case of AAV, capsid proteins.

A vector “genome” refers to the portion of the recombinant plasmid sequence that is ultimately packaged or encapsidated to form a viral (e.g., rAAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsidated into virus (e.g., rAAV) particles. Thus, a vector “genome” refers to the nucleic acid that is packaged or encapsidated by virus (e.g., rAAV).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Cross-reactivity differences are usually due to differences in capsid protein sequences/antigenic determinants (e.g., due to VP1, VP2, and/or VP3 sequence differences of AAV serotypes).

Under the traditional definition, a serotype means that the virus of interest has been tested against serum specific for all existing and characterized serotypes for neutralizing activity and no antibodies have been found that neutralize the virus of interest. As more naturally occurring virus isolates of are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

Recombinant vector (e.g., rAAV) include any viral strain or serotype. As a non-limiting example, a recombinant vector (e.g., AAV) plasmid or vector (e.g., AAV) genome or particle (capsid) can be based upon any AAV serotype, such as AAV-1, -2, -3, -4, -5, -6, -7, -8, -9, -10, -11, for example. Such vectors can be based on the same of strain or serotype (or subgroup or variant), or be different from each other. As a non-limiting example, a recombinant vector (e.g., rAAV) plasmid or vector (e.g., AAV) genome or particle (capsid) based upon one serotype genome can be identical to one or more of the capsid proteins that package the vector. In addition, a recombinant vector (e.g., AAV) plasmid or vector (e.g., AAV) genome can be based upon an AAV (e.g., AAV2) serotype genome distinct from one or more of the capsid proteins that package the vector genome, in which case at least one of the three capsid proteins could be a AAV1, AAV2, AAV3, AAV4, AAV5, AAV6, AAV7, AAV8, AAV9, AAV10, AAV 11, or variant thereof, for example. AAV vectors therefore include gene/protein sequences identical to gene/protein sequences characteristic for a particular serotype, as well as mixed serotypes.

In various exemplary embodiments, a rAAV vector includes or consists of a sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, or AAV11 capsid proteins. In various exemplary embodiments, a rAAV vector includes or consists of a sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, or AAV11 ITR(s).

Recombinant vectors (e.g., rAAV), such as AAV1, AAV2, AAV3, AAV4, AAV5, AAV6, AAV7, AAV8, AAV9, AAV10, and AAV11 and others, and variant, hybrid and chimeric sequences, can be constructed using recombinant techniques that are known to the skilled artisan, to include one or more heterologous polynucleotide sequences (transgenes) flanked with one or more functional AAV ITR sequences. Such vectors have one or more of the wild type AAV genes deleted in whole or in part, for example, a rep and/or cap gene, but retain at least one functional flanking ITR sequence, as necessary for the rescue, replication, and packaging of the recombinant vector into a rAAV vector particle. A rAAV vector genome would therefore include sequences required in cis for replication and packaging (e.g., functional ITR sequences)

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Nucleic acids include naturally occurring, synthetic, and intentionally modified or altered polynucleotides. Nucleic acids can be single, double, or triplex, linear or circular, and can be of any length. In discussing nucleic acids, a sequence or structure of a particular polynucleotide may be described herein according to the convention of providing the sequence in the 5′ to 3′ direction.

A “heterologous” nucleic acid sequence refers to a polynucleotide inserted into a vector (e.g., AAV) for purposes of vector mediated transfer/delivery of the polynucleotide into a cell. Heterologous nucleic acid sequences are typically distinct from vector (e.g., AAV) nucleic acid, i.e., are non-native with respect to viral (e.g., AAV) nucleic acid. Once transferred/delivered into the cell, a heterologous nucleic acid sequence, contained within the vector, can be expressed (e.g., transcribed, and translated if appropriate).

Alternatively, a transferred/delivered heterologous polynucleotide in a cell, contained within the vector, need not be expressed. Although the term “heterologous” is not always used herein in reference to nucleic acid sequences and polynucleotides, reference to a nucleic acid sequence or polynucleotide even in the absence of the modifier “heterologous” is intended to include heterologous nucleic acid sequences and polynucleotides in spite of the omission.

The “polypeptides,” “proteins” and “peptides” encoded by the “nucleic acid sequence,” include full-length native sequences, as with naturally occurring proteins, as well as functional subsequences, modified forms or sequence variants so long as the subsequence, modified form or variant retains some degree of functionality of the native full-length protein. Such polypeptides, proteins and peptides encoded by the nucleic acid sequences can be but are not required to be identical to the endogenous protein that is defective, or whose expression is insufficient, or deficient in the treated mammal.

A “transgene” is used herein to conveniently refer to a nucleic acid (e.g., heterologous) that is intended or has been introduced into a cell or organism. Transgenes include any nucleic acid, such as a heterologous nucleic acid encoding a therapeutic protein or polynucleotide sequence.

In a cell having a transgene, the transgene has been introduced/transferred by way of a plasmid or a vector, such as AAV, “transduction” or “transfection” of the cell. The terms “transduce” and “transfect” refer to introduction of a molecule such as a nucleic acid into a cell (e.g., HEK293) or host organism. The transgene may or may not be integrated into genomic nucleic acid of the recipient cell. If an introduced nucleic acid becomes integrated into the nucleic acid (genomic DNA) of the recipient cell or organism it can be stably maintained in that cell or organism and further passed on to or inherited by progeny cells or organisms of the recipient cell or organism.

A “transduced cell” is a cell into which a transgene has been introduced. Accordingly, a “transduced” cell means a genetic change in a cell following incorporation of an exogenous molecule, for example, a nucleic acid (e.g., a transgene) into the cell. Thus, a “transduced” cell is a cell into which, or a progeny thereof in which an exogenous nucleic acid has been introduced. The cell(s) can be propagated (cultured) and the introduced protein expressed or nucleic acid transcribed, or vector, such as rAAV, produced by the cell. For gene therapy uses and methods, a transduced cell can be in a subject.

As used herein, the term “stable” in reference to a cell, or “stably integrated” means that nucleic acid sequences, such as a selectable marker or heterologous nucleic acid sequence, or plasmid or vector has been inserted into a chromosome (e.g., by homologous recombination, non-homologous end joining, transfection, etc.) or is maintained in the recipient cell or host organism extrachromosomally, and has remained in the chromosome or is maintained extrachromosomally for a period of time. In the case of culture cells, nucleic acid sequences, such as a selectable marker or heterologous nucleic acid sequence, or plasmid or vector has been inserted into a chromosome can be maintained over the course of a plurality of cell passages.

An “expression control element” refers to nucleic acid sequence(s) that influence expression of an operably linked nucleic acid. Control elements, including expression control elements as set forth herein such as promoters and enhancers. Vector sequences including rAAV vectors can include one or more “expression control elements.” Typically, such elements are included to facilitate proper heterologous polynucleotide transcription and if appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but may also act in trans.

Expression control can be effected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances. Nevertheless, owing to the length limitations of certain vectors, such as rAAV vectors, expression control elements will typically be within 1 to 1000 nucleotides from the transcribed nucleic acid.

Functionally, expression of operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed sequence. A promoter typically increases an amount expressed from operably linked nucleic acid as compared to an amount expressed when no promoter exists.

An “enhancer” as used herein can refer to a sequence that is located adjacent to the nucleic acid sequence, such as selectable marker, or heterologous nucleic acid sequence Enhancer elements are typically located upstream of a promoter element but also function and can be located downstream of or within a sequence. Hence, an enhancer element can be located upstream or downstream, e.g., within 100 base pairs, 200 base pairs, or 300 or more base pairs of the as selectable marker, and/or a heterologous nucleic acid encoding a therapeutic protein or polynucleotide sequence Enhancer elements typically increase expression of an operably linked nucleic acid above expression afforded by a promoter element.

The term “operably linked” means that the regulatory sequences necessary for expression of a nucleic acid sequence are placed in the appropriate positions relative to the sequence so as to effect expression of the nucleic acid sequence. This same definition is sometimes applied to the arrangement of nucleic acid sequences and transcription control elements (e.g. promoters, enhancers, and termination elements) in an expression vector, e.g., rAAV vector.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (polyA) sequences) which flank a sequence, such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for vector packaging into a rAAV particle. In various embodiments, a filler/stuffer nucleic acid sequence is an untranslated (non-protein encoding) segment of nucleic acid. For a nucleic acid sequence less than 4.7 Kb, the filler or stuffer polynucleotide sequence has a length that when combined (e.g., inserted into a vector) with the sequence has a total length between about 3.0-5.5 Kb, or between about 4.0-5.0 Kb, or between about 4.3-4.8 Kb.

A “therapeutic protein” in one embodiment is a peptide or protein that may alleviate or reduce symptoms that result from an insufficient amount, absence or defect in a protein in a cell or subject. A “therapeutic” protein encoded by a transgene can confer a benefit to a subject, e.g., to correct a genetic defect, to correct a gene (expression or functional) deficiency, etc.

Non-limiting examples of heterologous nucleic acids encoding gene products (e.g., therapeutic proteins) which are useful in accordance with the invention include those that may be used in the treatment of a disease or disorder including, but not limited to, “hemostasis” or blood clotting disorders such as hemophilia A, hemophilia A patients with inhibitory antibodies, hemophilia B, deficiencies in coagulation Factors, VII, VIII, IX and X, XI, V, XII, II, von Willebrand factor, combined FV/FVIII deficiency, thalassemia, vitamin K epoxide reductase Cl deficiency, gamma-carboxylase deficiency; anemia, bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e. FXa inhibitors); and platelet disorders such as, Bernard Soulier syndrome, Glanzman thromblastemia, and storage pool deficiency.

Nucleic acid molecules, vectors such as cloning, expression vectors (e.g., vector genomes) and plasmids, may be prepared using recombinant DNA technology methods. The availability of nucleotide sequence information enables preparation of nucleic acid molecules by a variety of means. For example, a nucleic acid encoding Factor IX (FIX) can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

As disclosed herein, a way of producing recombinant viral vectors such as rAAV vectors according to the invention is to express heterologous nucleic acid encoding a therapeutic protein or polynucleotide in an invention HEK (e.g., HEK293) cell or cell line, human A459 cell or cell line and/or Vero cell or cell line. The cell or cell line will provide helper functions for viral (e.g., AAV) vector packaging and produce rAAV under appropriate culture conditions. Accordingly, the invention provides HEK (e.g., HEK293) cell and cell line, human A459 cell and cell line and/or Vero cell and cell line that produce recombinant viral vectors such as rAAV vectors as well as methods of producing recombinant viral vectors such as rAAV vectors. The invention HEK (e.g., HEK293) cell or cell line, human A459 cell or cell line and/or Vero cell or cell line include expression of heterologous nucleic acid as well as providing helper functions for viral (e.g., AAV) vector packaging. The method including expression of heterologous nucleic acid in an invention HEK (e.g., HEK293) cell or cell line, human A459 cell or cell line and/or Vero cell or cell line that provides helper functions for viral (e.g., AAV) vector packaging.

As also disclosed herein, a way of producing recombinant proteins according to the invention is to express nucleic acid encoding such protein(s) in an invention HEK (e.g., HEK293) cell or cell line, human A459 cell or cell line and/or Vero cell or cell line. Accordingly, the invention also provides cells that produce recombinant proteins as well as methods of making recombinant proteins. The invention HEK (e.g., HEK293) cell or cell line, human A459 cell or cell line and/or Vero cell or cell line include expression of nucleic acid encoding recombinant protein. The method including expression of nucleic acid encoding recombinant protein in an invention HEK (e.g., HEK293) cell or cell line, human A459 cell or cell line and/or Vero cell or cell line.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane.

With respect to protein, the term “isolated protein” or “isolated and purified protein” is sometimes used herein. This term refers primarily to a protein produced by expression of a nucleic acid molecule. Alternatively, this term may refer to a protein which has been sufficiently separated from other proteins with which it would naturally be associated, so as to exist in “substantially pure” form.

The term “isolated” does not exclude combinations produced by the hand of man, for example, a recombinant vector sequence, or virus particle that packages or encapsidates a vector genome (e.g., rAAV) and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The phrase “consisting essentially of” when referring to a particular nucleotide sequence or amino acid sequence means a sequence having the properties of a given sequence. For example, when used in reference to an amino acid sequence, the phrase includes the sequence per se and molecular modifications that would not affect the basic and novel characteristics of the sequence.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features (e.g., nucleic acid sequences, vectors, viral vectors, rAAV vectors, etc.) are an example of a genus of equivalent or similar features.

As used herein, the singular forms “a”, “and,” and “the” include plural referents unless the context clearly indicates otherwise. Thus, for example, reference to “a nucleic acid sequence,” or “selectable marker,” includes a plurality of such nucleic acid sequences and selectable markers, and reference to “a vector” includes a plurality of such vectors, such as rAAV vectors.

As used herein, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges are inclusive. Further, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed.

### EXAMPLES

### Example 1

This example describes producing invention HEK cells and cell lines, and subsequent transfer of viral genomes and virus (AAV) vector production.

Invention HEK cells and cell lines can be produced in a variety of ways, by knocking out the cell's endogenous DHFR gene and/or GS gene. For example, certain non-limiting methods include Zinc-finger nucleases (ZFNs) for targeted cleavage and gene inactivation (See, e.g., United States Patent Publications 20030232410; 20050208489; 20050026157; 20050064474; 20060188987; 20060063231; 2008/0015164; and International Publication WO 07/014275). ZFNs provide the ability to place a double-strand DNA break (DSB) at a chosen genomic address. The removal of this site-specific DSB is carried out by the cell's own DNA repair machinery via a homology-directed repair process when donor DNA is provided, or via non-homologous end joining (NHEJ)—See, e.g., Urnov et al. (2005) Nature 435:646-651 (2005); Moehle et al. (2007) Proc Natl Acad Sci USA 104:3055-3060 (2007); Bibikova, et al. (2001) Mol Cell Biol 21:289-297; Bibikova et al. (2003) Science 300:764; Porteus et al. (2005) Nature Biotechnology 23:967-973; Lombardo et al. (2007) Nature Biotechnology 25:1298-1306; Perez et al. (2008) Nature Biotechnology 26:808-816; Bibikova et al. (2002) Genetics 161:1169-1175; Lloyd et al. (2005) Proc Natl Acad Sci USA 102:2232-2237; Morton et al. (2006) Proc Natl Acad Sci USA 103:16370-16375.

CRISPR/Cas9 editing is another method that can be used to produce invention HEK, human A459 and/or Vero cells, cell lines and cell clones with reduced expression of endogenous DHFR and/or GS or knocked out endogenous DHFR gene and/or GS gene. CRISPR/Cas9 system for targeted gene modification/deletion has been described extensively (See, e.g., Qi L S, et al. Cell. 152(5), 1173-1183 (2013); Cong L, et al. Science. 339(6121), 819-823 (2013); Hsu P D, et al. Cell. 157(6), 1262-1278 (2014); Hsu P D, et al. Nat Biotechnol. 31(9), 827-832 (2013); and Doudna J A, Charpentier E. Science. 346(6213), 1258096 (2014).

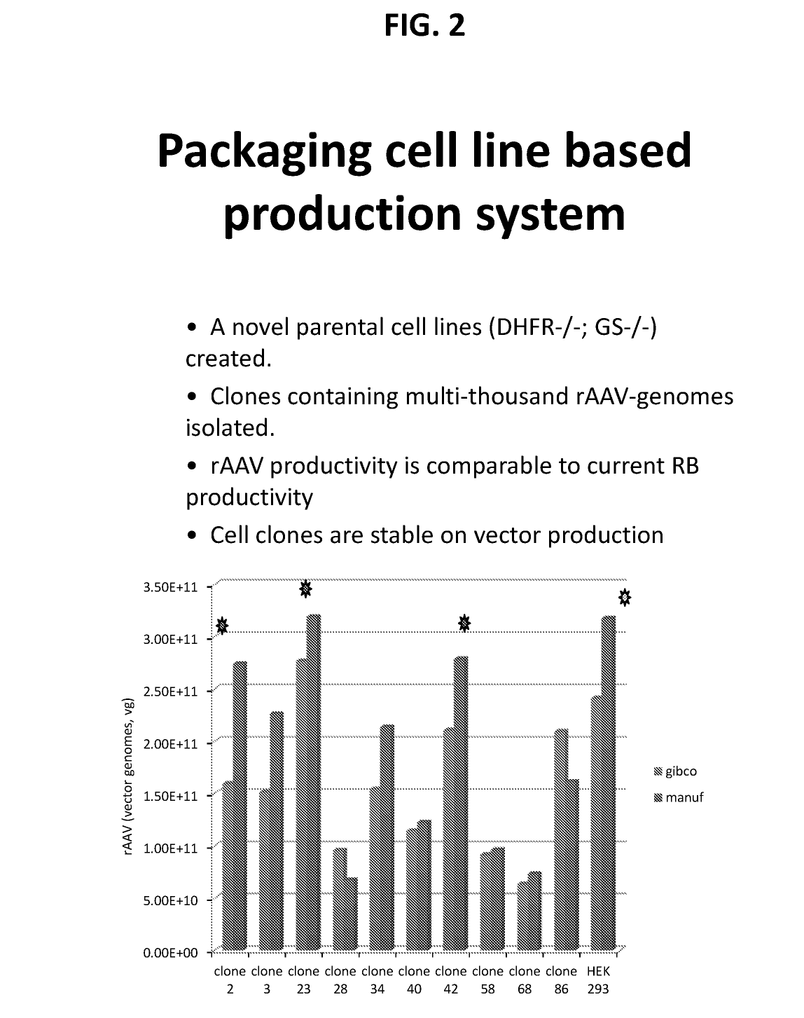

rAAV-hFix construct associated with either DHFR gene expression cassette or GS expression cassette was constructed and transfected into the HEK293 DHFR−/−/GS−/− cell line. Stable clones were isolated using MTX or MSX as selection markers. Clones contain high copy numbers of rAAV-hFiX genome and producing more rAAV vectors were maintained for further characterization. A number of isolated cell clones were stable and demonstrated high AAV production (FIG. 2).

### Example 2

This example describes certain non-limiting features of invention HEK cells and cell lines.

For example, in general:
A. Stable producer clones.
B. Human mammalian manufacturing cell clones with DHFR and/or GS gene(s) knocked out will enable selection of stable clones to express gene(s) of interest using DHFR or GS genes (or both) as selection markers, clones can be create for any gene(s) of interest, or viral vectors.
C. Safer producing human cell line suitable for manufacture bio-products with gene amplification function to provide high productivity.
D. DHFR and/or GS negative genome background of cell lines created will allow gene amplification of the gene(s) of interest, therefor leads to high specific productivity.
E. Clean genomic background, no need to introduce antibiotic markers to select positive producer clones, therefor safer clones to manufacture recombinant proteins, viral vectors such as rAAV vectors.
F. Reduced labor cost and material cost significantly.

For example, for rAAV production:
A. Increased rAAV yield
B. Easy to scale up for production of large quantities/large scale rAAV production.
C. Safer production system in comparison helper virus involved production system, such as production systems using adenovirus as helper or Baculovirus based production systems.
D. Produce rAAV vectors with low amount of empty capsids by reducing empty particles and reduce DNA impurities packed in rAAV vectors.
E. Achieved high copy numbers of rAAV genomes for certain cell clones, high rAAVhFix producer clones identified.
F. DHFR/GS double knock in with rAAVhFix genome allows substantial amplification of rAAVhFix genome, therefor leads to high rAAV production.

For example, for protein production:
A. The HEK, human A459 and/or Vero cells and cell lines may fold and modify bio-products closer to its native conditions as produce in human, therefor the products produced from the HEK, human A459 and/or Vero cells and cell lines will be safer and more potent.
B. Bio-products produced from HEK (e.g., HEK293DHFR−/−/GS−/− cell clones created), and/or human A459 will enable post translational modification closer to its natural products from human body since HEK (e.g., HEK293) and A549 cells are human cell lines, not another non-human species.

What is claimed is:

1. A human embryonic kidney (HEK) cell which does not express functional endogenous di-hydrofolate reductase (DHFR) and/or glutamine synthetase (GS).

2. A human embryonic kidney (HEK) cell line which does not express functional endogenous di-hydrofolate reductase (DHFR) and/or glutamine synthetase (GS).

3. The HEK cell or cell line of claim 1 or 2, stably or transiently transfected with a first heterologous nucleic acid sequence, and optionally stably or transiently transfected with a second heterologous nucleic acid sequence.

4. The HEK cell or cell line of claim 1 or 2, stably or transiently transfected with a first heterologous nucleic acid sequence and a first selectable marker, and optionally stably or transiently transfected with a second heterologous nucleic acid sequence and a second selectable marker.

5. The HEK cell or cell line of claim 3 or 4, wherein the first heterologous nucleic acid sequence encodes a therapeutic protein or polynucleotide sequence and optional second heterologous nucleic acid sequence encodes a therapeutic protein or polynucleotide sequence.

6. The HEK cell or cell line of claim 4, wherein the therapeutic protein or polynucleotide sequence encoded by the first heterologous nucleic acid sequence and the therapeutic protein or polynucleotide sequence encoded by the optional second heterologous nucleic acid sequence are the same or are different.

7. The HEK cell or cell line of claim 4, wherein the first or second selectable marker does not provide resistance to an antibiotic.

8. The HEK cell or cell line of claim 4, wherein the first or second selectable marker provides a means to amplify the first and/or second heterologous nucleic acid sequence(s).

9. The HEK cell or cell line of claim 4, wherein the first or second selectable marker comprises a nucleic acid encoding a protein having DHFR function.

10. The HEK cell or cell line of claim 4, wherein the first or second selectable marker comprises a nucleic acid encoding a protein having GS function.

11. The HEK cell or cell line of claim 4, wherein the first selectable marker comprises a nucleic acid encoding a protein having DHFR function and the second selectable marker comprises a nucleic acid encoding a protein having GS function.

12. The HEK cell or cell line of claims 3 to 11, wherein the first heterologous nucleic acid sequence comprises a first vector, and the optional second heterologous nucleic acid sequence comprises a second vector.

13. The HEK cell or cell line of claim 11 or 12, wherein the first vector and optional second vector are the same or are different.

14. The HEK cell or cell line of claim 11 or 12, wherein the first vector and optional second vector each comprises a selectable marker comprising a nucleic acid encoding a protein having DHFR function or a nucleic acid encoding a protein having GS function.

15. The HEK cell or cell line of any of claims 12-14, wherein the first vector comprises a first viral vector and optional second vector comprises a second viral vector.

16. The HEK cell or cell line of any of claims 12-14, wherein the first or second viral vector comprises an AAV vector genome.

17. The HEK cell or cell line of any of claims 12-14, comprising first and second viral vectors, wherein the viral vectors each comprise an AAV vector genome

18. The HEK cell or cell line of claim 17, wherein the AAV vector genome(s) comprises one or two AAV ITRs that flank the 5′ and/or 3′ ends of the heterologous nucleic acid sequence.

19. The HEK cell or cell line of any of claims 4-18, wherein copy number of the heterologous nucleic acid sequence(s) and/or vector(s) and/or viral vector(s) and/or AAV vector genome(s) in the HEK cell or cell line is 1-5 copies per cell, 5-10 copies per cell, 10-50 copies/cell, 50-100 copies per cell, 100-250 copies per cell, 250-500 copies per cell, 500-1,000 copies per cell, 1,000-2,000 copies per cell, or about or greater than 2,000, 3,000, 4,000 or 5,000 copies per cell, and optionally wherein copy number appears stable over many passages, e.g., at least or greater than 5, 10, 15 or more passages.

20. The HEK cell or cell line of any of claims 16-19, wherein copy number of the AAV vector genome(s) in the HEK cell or cell line is at least 1,000 copies per cell, and the rAAV vector particle yield optionally is at least 1×10⁸ vg/ml, or at least 1×10⁹ vg/ml, or at least 1×10¹⁰ vg/ml, or at least 1×10¹¹ vg/ml or at least 2×10¹¹ vg/ml from a Roller Bottle of HEK cell or the HEK cell line.

21. The HEK cell or cell line of any of claims 1-20, further comprising AAV rep and/or cap sequences.

22. The HEK cell or cell line of claim 20, wherein the AAV rep and/or cap sequences are provided by a plasmid that either transiently or stably transfected into the HEK cell or cell line.

23. The HEK cell or cell line of any of claims 1-22, further comprising AAV helper function sequences.

24. The HEK cell or cell line of claim of any of claims 1-23, wherein the HEK cells or cell line are HEK 293.

25. The HEK cell or cell line of any of claims 1-24, in a culture or growth medium, or in a medium suitable for long-term storage.

26. The HEK cell or cell line of any of claims 9-25, in a culture medium or growth comprising methotrexate (MTX) and/or methionine sulphoxamine (MSX).

27. The HEK cell or cell line of any of claims 6-26, wherein the HEK cell or cell line produces rAAV vector particles having packaged therein the heterologous nucleic acid sequence(s).

28. The HEK cell or cell line of claim 27, wherein the rAAV vector particles are produced in greater amounts than amounts produced by HEK293 cells that express functional endogenous DHFR and/or GS and transiently transfected with AAV vector genomes having the heterologous nucleic acid sequence.

29. The HEK cell or cell line of claim 27, wherein the AAV vector particles produced contain less quantities of rAAV empty capsids and/or less quantities of rAAV particles that have packaged contaminating DNA than amounts of AAV empty capsid and/or rAAV particles that have packaged contaminating DNA produced by HEK293 cells that express functional endogenous DHFR and/or GS and transiently transfected with rAAV vector genomes having the heterologous nucleic acid sequence.

30. The HEK cell or cell line of any of claims 3-29, wherein the heterologous nucleic acid sequence(s) encodes a therapeutic protein(s) or an inhibitory nucleic acid sequence(s).

31. The HEK cell or cell line of claim 30, wherein the therapeutic protein(s) comprises a blood clotting factor or immunoglobulin sequence.

32. The HEK cell or cell line of claim 30, wherein the inhibitory nucleic acid sequence comprises a small or short hairpin (sh)RNA, microRNA (miRNA), small or short interfering (si)RNA, trans-splicing RNA, or antisense RNA.

33. The HEK cell or cell line of any of claims 3-32, stably transfected with the first heterologous nucleic acid sequence.

34. The HEK cell or cell line of any of claims 3-32, stably transfected with the first and the second heterologous nucleic acid sequence.

35. Viral or rAAV vector particles isolated or purified from the HEK cell or cell line of any of claims 15-34.

36. Therapeutic protein(s) isolated or purified from the HEK cell or cell line of any of claims 5-34.

37. A method of producing a therapeutic protein(s), viral vector(s) or rAAV vector particles comprising culturing the HEK cell or cell line of any of claims 5-34, under conditions allowing production and/or secretion of the therapeutic protein(s), viral vector(s) or rAAV vector particles and isolating or purifying the therapeutic protein(s), viral vector(s) or rAAV vector particles from the cell culture, culture medium, or cell culture and culture medium.

38. A method of producing a rAAV vector particles comprising culturing the HEK cell or cell line of any of claims 23-25, under conditions allowing production and/or secretion of the rAAV vector particles and isolating or purifying the rAAV vector particles from the cell culture, culture medium, or cell culture and culture medium, wherein when the HEK cell or cell line has at least 1,000 copies per cell of AAV vector genome the rAAV vector particle yield is at least 2×10¹¹ vg/Roller Bottle of HEK cell or the HEK cell line.

39. The HEK cell or cell line method according to any of claims 3-38, wherein said first and/or second heterologous nucleic acid sequence encodes a gene product selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), TGFβ, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase.

40. The HEK cell or cell line or method according to any of claim 23-29, 37 or 38, wherein said rAAV vector particles comprise the first and/or the second heterologous nucleic acid sequence encoding a gene product selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), TGFβ, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase.

41. The HEK cell or cell line method according to any of claims 3-38, wherein said first and/or second heterologous nucleic acid sequence encodes a gene product selected from the group consisting of thrombopoietin (TPO), interleukins (IL1 through IL-17), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

42. The HEK cell or cell line or method according to any of claim 23-29, 37 or 38, wherein said rAAV vector particles comprise the first and/or the second heterologous nucleic acid sequence encoding a gene product selected from the group consisting of thrombopoietin (TPO), interleukins (IL1 through IL-17), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

43. The HEK cell or cell line method according to any of claims 3-38, wherein said first and/or second heterologous nucleic acid sequence encodes a protein useful for correction of in born errors selected from the group consisting of carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor V, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, RPE65, H-protein, T-protein, a cystic fibrosis transmembrane regulator (CFTR) sequence, and a dystrophin cDNA sequence.

44. The HEK cell or cell line or method according to any of claim 23-29, 37 or 38, wherein said rAAV vector particles comprise the first and/or the second heterologous nucleic acid sequence encoding encodes a protein useful for correction of in born errors selected from the group consisting of carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor V, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, RPE65, H-protein, T-protein, a cystic fibrosis transmembrane regulator (CFTR) sequence, and a dystrophin cDNA sequence.

45. A method of producing a human embryonic kidney (HEK) cell line which does not express functional endogenous di-hydrofolate reductase (DHFR) comprising mutating or knocking out the endogenous DHFR gene.

46. A method of producing a human embryonic kidney (HEK) cell line which does not express functional endogenous glutamine synthetase (GS), comprising mutating or knocking out the endogenous GS gene.

47. A method of producing a human embryonic kidney (HEK) cell line which does not express functional endogenous di-hydrofolate reductase (DHFR) and glutamine synthetase (GS), comprising mutating or knocking out the endogenous DHFR gene and GS gene.
